(19) United States
(12) Patent Application Publication
(10) Pub. No.: US 2013/0109034 A1
(43) Pub. Date: May 2, 2013

(54) Compositions and Methods for Determining Celiac Disease

(71) Applicants: Vijay Kumar, Amherst, NY (US); Kishore S. Malyavantham, Williamsville, NY (US)

(72) Inventors: Vijay Kumar, Amherst, NY (US); Kishore S. Malyavantham, Williamsville, NY (US)

(73) Assignee: IMMCO DIAGNOSTICS, INC., Buffalo, NY (US)

(21) Appl. No.: 13/583,125

(22) PCT Filed: Mar. 10, 2011
(86) PCT No.: PCT/US11/27865

§ 371 Date: Jan. 16, 2013

Related U.S. Application Data
(60) Provisional application No. 61/312,393, filed on Mar. 10, 2010.

(51) Int. Cl. G01N 33/68; C12N 9/96

(52) U.S. Cl. CPC G01N 33/6854; C12N 9/96
USPC 435/7.92; 435/188

(57) ABSTRACT

Provided are compositions and methods for diagnosis of celiac disease. The compositions include recombinant proteins that contain tissue transglutaminase and deamidated gliadin sequences. The gliadin sequences are repeated in the recombinant proteins. Also provided is a method for identifying an individual as having celiac disease based on the presence of antibodies in a sample from the individual, where the anti-bodies specifically recognize the recombinant protein, and identifying the individual as not having celiac disease based on an absence of antibodies that specifically recognize the recombinant protein.

### CROSS-REFERENCE TO RELATED APPLICATION

This application claims priority to U.S. application Ser. No. 61/312,393, filed Mar. 10, 2010, the disclosure of which is incorporated herein by reference.

### FIELD OF THE INVENTION

The present invention relates generally to the field of celiac disease. More particularly, the invention provides improved compositions and methods for detecting celiac disease.

### BACKGROUND OF THE INVENTION

Celiac Disease (CD) is an autoimmune gastrointestinal disorder that may occur in genetically susceptible individuals triggered by the ingestion of gluten-containing grains such as wheat, barley and rye. Of the many autoimmune disorders, CD represents one of the few disorders where the etiological agent is known and the disease subsides and goes in remission once the etiological agent is withdrawn from the diet. CD is characterized by malabsorbtion resulting from inflammatory injury to the small intestinal mucosa and, when prolonged, can cause malnutrition. The classical symptoms of CD include diarrhea, weight loss and malnutrition. Only a small percentage of patients with CD, however, presents with classical symptoms. Consequently, the clinical spectrum of CD has grown much broader than in the past to include patients that do not present with classical symptoms. It is not uncommon for the initial symptoms to be non-gastrointestinal or for gastrointestinal symptoms, if present, to be mild or intermittent. Studies have found the prevalence of CD to be approximately 1%. If only the clinical criteria are used in determining prevalence, the incidence of CD is much lower as compared with incidence established by serological methods, suggesting that many subjects may present with non-classical or are asymptomatic.

Failure to diagnose CD early on may predispose an individual to long-term complications such as splenic atrophy and intestinal lymphoma. In a recent study, CD was shown to be associated with significantly elevated risk for intestinal lymphoma, especially for non-Hodgkin lymphoma. Some serological methods for the detection of antibodies to gliadin, endomysium and tissue transglutaminase have permitted screening for CD.

Because of the varied clinical presentations and the increase incidence of CD in the general population, it is very desirable to have an immunoassay that is sensitive yet specific. The limitations of the existing immunoassays include: a) limited specificity. Many of the instrument immunoassays such as for tTG and gliadin antibodies have limited specificities as antibodies to tTG and to gliadin have been reported in subjects with no celiac disease such as in primary biliary cirrhosis, rheumatoid arthritis, ankolysing spondlylitis, Sjogren's syndrome etc. Several studies have reported that the positive findings in many of such cases are false positive. b) Sensitivity. The sensitivity of many of these assays is about 95% at the best and hence many patients with CD may be reported as negative. c) False negatives in patients with CD who are IgA deficient. The existing immunoassays detect IgA class antibodies to tTG, gliadin and EMA and hence such subjects with IgA deficiency may be reported as negative. There is an urgent need to have immunoassays that can detect CD with IgA deficiency. There are immunoassays that can detect IgG antibodies to tTG and to gliadin. However the specificity of these methods is questionable. Hence, methods that are specific, sensitive and that can also detect IgA deficient CD cases with reliability are highly desirable. Further, the current practice for CD diagnosis is to use a combination of various diagnostic methods. However, the use of multiple assays not only is expensive but also results in discrepancies due to limitations of the assays in specificity and sensitivity. Thus, there is an ongoing need for improved compositions and methods for diagnosing CD.

### SUMMARY OF THE INVENTION

The present invention provides compositions and methods for diagnosing celiac disease. The method comprises determining from a biological sample obtained and/or derived from an individual the presence or absence of antibodies that specifically recognize a chimeric protein that contains tissue transglutaminase (tTG) and deamidated gliadin amino acid sequences (DGP: deamidated gliadin peptide), the latter of which are repeated in the chimeric protein. The presence of antibodies that specifically recognize the chimeric protein is indicative that the individual has celiac disease; the absence of antibodies that specifically recognize the chimeric protein is indicative that the individual does not have celiac disease.

The chimeric proteins provided by the invention comprise an amino acid sequence of tissue transglutaminases (tTG) and the amino acid sequence of deamidated gliadin. The amino acid sequence of the deamidated gliadin is repeated in the chimeric protein between three and seven times. In one embodiment, the deamidated gliadin is repeated three times. In another embodiment, the deamidated gliadin is repeated five times.

The invention is particularly efficient at discriminating the presence or absence of IgG and IgA antibodies that recognize the chimeric protein and are accordingly indicative of CD. In one embodiment, the individual who is identified as having CD according to the invention is an IgA deficient individual.

Also provided are chimeric proteins of the invention and compositions comprising them. The chimeric proteins that can be used in the method of the invention can contain repeated complete deamidated gliadin sequences or repeated segments of the deamidated gliadin. The deamindated gliadin sequences may occur in series, with or without linker or longer sequences between them, and can be present anywhere in the chimeric protein, including but not limited to the N-terminus, the C-terminus, or anywhere between the N- and C-terminus of the chimeric protein.

### BRIEF DESCRIPTION OF THE FIGURES

FIG. 1 provides a graphical illustration of a DNA construct encoding a chimeric protein provided by the invention, as well as an amino acid sequence of a representative chimeric protein that is suitable for use in the method of the invention (SEQ ID NO:1). A representative amino acid sequence of tTG that can be used in the invention is presented in amino acids 52-739 of SEQ ID NO:1. Amino acids 4-9 comprise a HIS tag. Amino acids 13-21 comprise a deamidated gliadin peptide, which is repeated two additional times in SEQ ID NO:1. The SSG, GSG and amino acids 46-51 comprises spacers.

FIG. 2 provides a graphical depiction of a cloning scheme for a chimeric protein of the invention, as well as a photographic representation of an electrophoretic analysis of a recombinant expression vector suitable for making a chimeric protein of the invention.

FIG. 3 provides a photographic representation of the expression and purification of a chimeric protein of the invention. The chimeric protein comprises the amino acid sequence shown in FIG. 1.

FIG. 4 provides a tabular summary obtained by analysis of clinical samples from patients with CD to detect IgA and IgG antibodies that recognize a chimeric protein having the amino acid sequence depicted in FIG. 1.

FIG. 5 provides a graphical representation of data obtained from a comparison of detecting autoantibodies in sera of patients with CD and who are positive for endomysial antibodies. There is a noteworthy advantage provided by use of the chimeric molecule for detecting antibodies when the EMA levels are low.

FIG. 6 provides a graphical analysis of data obtained from ELISA assays of antibody positive sera using a chimeric protein of the invention that comprises 1, 3 or 5 deamidated gliadin peptide (DGP) repeats.

FIG. 7 provides a graphical summary of data that illustrates the sensitivity of the assays provided by the invention. The sensitivity of the screen assay that uses tTG (tissue transglutaminase)-5X-DGP (deamidated gliadin peptide) fusion for IgA and IgG antibodies is higher than combined sensitivities of individual tTG assays for IgA and DGP assay for IgG respectively. The enhanced sensitivity of the invention is conspicuous against the sera with lower EMA (antibodies against the endomysial antigen) titers.

FIG. 8 provides a graphical summary of data that illustrate the specificity of chimeric tTG 5x DGP (i.e: comprising five repeats of the deamidated gliadin peptide equivalent) screen assay for both IgA and IgG antibodies versus combined specificities of individual tTG and DGP assays for IgA and IgG respectively.

### DESCRIPTION OF THE INVENTION

The present invention provides compositions and methods for diagnosis of celiac disease. The compositions include chimeric proteins that comprise the amino acid sequence of tissue transglutaminases (tTG) and the amino acid sequence of deamidated gliadin, wherein the amino acid sequence of gliadin is repeated in the chimeric protein. Polynucleotides encoding the chimeric proteins are also included within the scope of the invention.

The method of the invention comprises contacting a biological sample obtained and/or derived from an individual with a chimeric protein of the invention and detecting the presence or absence of antibodies that specifically recognize the chimeric protein. The presence of antibodies that specifically recognize the chimeric protein is indicative that the individual has celiac disease. The absence of antibodies is indicative that the individual does not have celiac disease. Thus, the individual can be identified as having or not having celiac disease based on the presence or absence of the antibodies, respectively. In one embodiment, the “absence” of antibodies can be evidenced by signal from an immunodiagnostic assay where the signal does not differ from a control or background signal in a statistically significant way. In one embodiment, this immunodiagnostic assay is an ELISA assay.

The advantages of the present method include but are not necessary limited to 1) elimination of the requirement for the use of multiple, distinct assays in diagnosing CD; 2) both IgA and IgG class antibodies are detected, which enables the identification of subjects with CD who are IgA deficient; and 3) the presently provided immunoassay are more sensitive due to the detection of both tTG and deamidated gliadin antibodies using a single chimeric protein in the immunoassays. In particular, when tested under identical conditions for antibodies to tTG alone, to deamidated gliadin peptides alone, and to the presently provided chimeric protein, the results show improved results when using the chimeric protein. Specifically, if a clinical sample is IgA or IgG positive for antibodies to deamidated gliadin peptides and tTG in both assays, it is also positive in a single assay performed according to the method of the invention. Further, if a sample is negative for deamidated gliadin peptide and tTG, it is also negative when analyzed according to the present invention. Thus, the invention provides superior compositions and methods for diagnosing CD than were previously available. In one embodiment, the invention provides superior detection of IgA antibodies, relative to analyzing a biological sample using distinct tTG and deamindated gliadin molecules in separate assays screening for IgA and IgG antibodies respectively. Thus, the invention is particularly suitable for diagnosing CD in individuals who are IgA deficient with the help of a single quantitative immunoassay. In one embodiment, the invention is used for diagnosis of CD in individuals who exhibit an endoymysial antigen (EMA) titer in the range of 2.5 to 40, inclusive, and including all digits to the tenth decimal place there between. The present invention has the ability to quantitatively screen for both IgA and IgG antibodies that are specific for tTG and DGP epitopes by a single assay which can further replace the screening strategy for CD by traditional immunofluorescence based technique intensive EMA staining IgA antibodies.

In one embodiment, the chimeric proteins of the invention comprises at least one tTG amino acid sequence fused to at least three deamidated gliadin amino acid sequences, wherein the at least one tTG sequence is present in a contiguous polypeptide with the at least three deamidated gliadin sequences. The phrase “tTG amino acid sequence” means the amino acid sequence of which a tTG protein is comprised. The phrase “deamidated gliadin amino acid sequence” means the amino acid sequence of which a deamidated gliadin peptide is comprised.

In one embodiment, the tTG amino acid sequence and deamidated gliadin amino acid sequences are linked via a series of peptide bonds. It will therefore be recognized that, in one embodiment, the tTG amino acid sequence and the at least three deamidated gliadin amino acid sequences are not linked to one another via a covalent bond that was formed by a chemical cross-linking agent. Accordingly, in certain embodiments, the chimeric proteins used in the method of the invention are distinct from tTG/gliadin complexes formed by non-covalent interactions and from tTG/gliadin complexes formed by cross-linking agents. Notwithstanding the foregoing, there is no limitation on whether the chimeric protein itself can be cross-linked or otherwise connected to other compositions of matter, one non-limiting example of which is a solid matrix in an immunological assay system or device.

The number of tTG and deamidated gliadin amino acid sequences that are present in the chimeric proteins of the invention is variable. For example, the invention includes chimeric proteins that, in addition to at least one tTG amino acid sequence, include 3, 4, 5, 6, 7 or more deamidated gliadin amino acid sequences. In one embodiment, the chimeric protein comprises only three gliadin amino acid sequences and only one tTG amino acid sequence.

With respect to deamidated proteins, it will be recognized by those skilled in the art that deamidated proteins are those that have had some or all of the free amide functional groups hydrolyzed to carboxylic acids, such as conversion of glutamines to glutamic acid. Thus, a deamidated amino acid sequence can comprise replacement of glutamines with glutamic acids.

In one embodiment, the deamidated gliadin amino acid sequence in the chimeric protein comprises or consists of peptide sequences of Gliadin protein consisting of immuno-reactive regions of either the loop regions or the helices or the beta sheets of the three dimensional structure or other water accessible areas of the Gliadin protein.

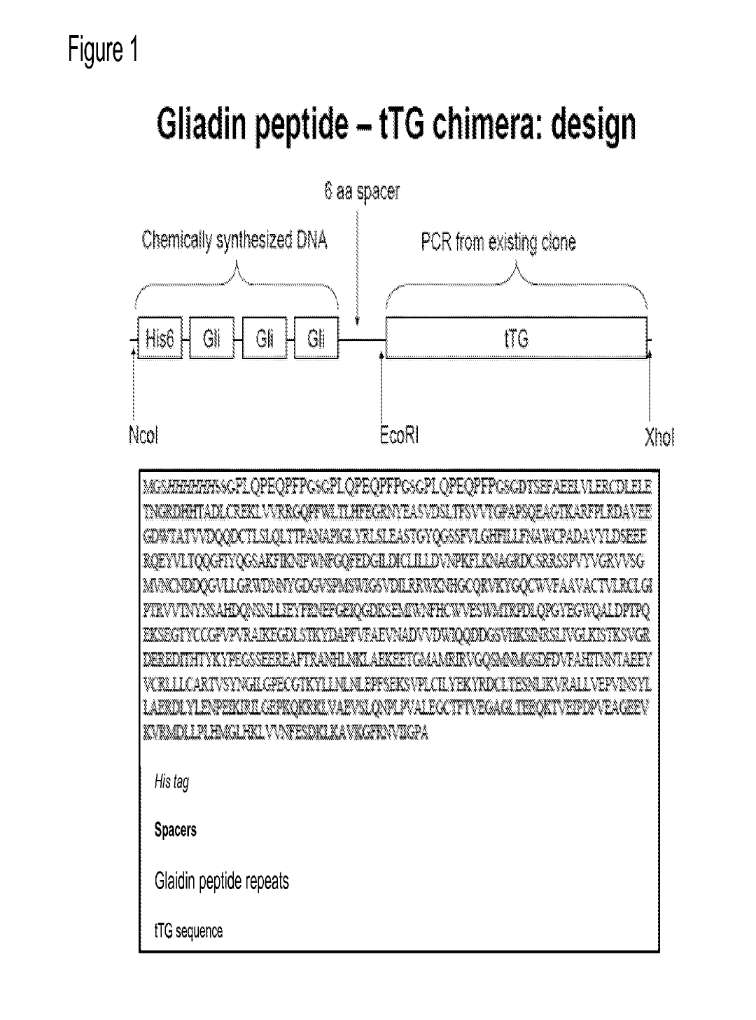

In one embodiment, the deamidated gliadin amino acid sequence in the chimeric protein comprises or consists of the sequence PLQPEQPFP (SEQ ID NO:2). In one embodiment, the tTG amino acid sequence in the chimeric protein comprises or consists of the tTG amino acid sequence depicted in FIG. 1. In FIG. 1, amino acids 1-3 and from 52-739, inclusive, constitute a tTG sequence.

Any gliadin amino acid sequence in the chimeric protein used in the invention can be completely contiguous with another gliadin amino acid sequence, or individual gliadin amino acid sequences can be separated by a linker or spacer peptide consisting of one or more non-gliadin amino acids. For example, a spacer sequence can be present between gliadin amino acid sequences. The spacer sequence can be 1, 2, 3, 4, 5, 6 or more amino acids in length. In various embodiments, the spacer comprises a serine, a glycine, or a combination thereof, and/or these and other amino acids.

One or more of the at least three gliadin amino acid sequences can be present at the N-terminus or the C-terminus of the chimeric protein used in the invention. In an alternative embodiment, the gliadin sequences can intersperse the tTG amino acid sequence at one or more times, in contiguous or non-contiguous locations. Alternatively, any or all of the gliadin amino acid sequences can be flanked at one or both ends by other amino acids. In one embodiment, a gliadin amino acid sequence is contiguous with an amino acid sequence that is used to facilitate synthesis and/or purification of the chimeric protein. For example, one of the gliadin amino acid sequences can be immediately adjacent to a HIS tag, or could be separated from a HIS tag by spacer or other amino acid sequence(s). In another embodiment, an amino acid sequence that is used to facilitate synthesis and/or purification of the chimeric protein is immediately adjacent to the tTG amino acid sequence, or is separated from the tTG amino acid sequence by gliadin or non-gliadin amino acid sequences.

The chimeric proteins of the invention can be made using techniques well known to those skilled in the art. For example, any DNA sequence encoding a chimeric protein of the invention can be made using standard techniques and inserted into any number of expression vectors. Suitable expression vectors have been described in the literature and many are commercially available. Likewise, a wide variety of expression systems are known in the art and are commercially available, including prokaryotic and eukaryotic systems. Chemical synthesis may also be used to make the chimeric proteins. The chimeric protein can be isolated from the expression system and purified for use in the invention to any desired degree of purity.

The biological sample obtained from the individual can be any biological sample that comprises antibodies, and can comprise biological tissue and/or biological liquid. It is preferable to use a biological liquid obtained from the individual. In one embodiment, the biological liquid is blood or serum. In other embodiments, the biological liquid is mucous, urine, lymph, fecal material, intestinal lavage or rectal effluents. The biological sample that is obtained from the individual can be used directly in determining the presence or absence of the antibodies, or the biological sample can be subjected to one or more processing steps before the biological sample is used in determining the presence or absence of the antibodies.

The chimeric protein can be used in the method of the invention in association with any composition of matter, device or system that is suitable for detecting antibodies. In one embodiment, the chimeric protein is in physical association with a solid matrix. The solid matrix may be present in a multi-well assay plate, beads, a lateral flow device or strip, or any other form or format that is suitable for keeping the chimeric protein in a position whereby antibodies can bind to it and be detected in the method of the invention. The chimeric protein may be covalently or non-covalently associated with the solid matrix.

Any technique, device, system and/or reagents can be used to detect antibodies in the biological sample when performing the invention. In one non-limiting example, antibodies can be detected using an Enzyme-linked immunosorbent assay (ELISA), or any modification of an ELISA that is suitable for detecting antibodies. Further, any isotype of antibody can be detected in the method of the invention. Non-limiting examples of antibody isotypes that can be detected and discriminated from one another by performing the invention are IgG and IgA antibodies.

In one embodiment, the invention provides a composition comprising a chimeric protein of the invention, wherein the chimeric protein is present in a complex with antibodies that specifically recognize the chimeric protein. In one embodiment, the antibodies in the complex are in or from a biological sample obtained or derived from a CD patient. Thus, in one embodiment, the invention provides a composition comprising antibodies that have been isolated from a CD patient, wherein the antibodies are complexed (e.g., specifically bound to) a chimeric protein of the invention. In one embodiment, the antibody/chimeric protein complex comprises IgA antibodies from the individual.

Relative to previously available assays, the invention provides for increased sensitivity, increased specificity, or a combination thereof, when detecting antibodies that specifically recognize tTG or deamidated gliadin.

In one embodiment, the invention facilitates detection of the presence or absence of antibodies that specifically recognize tTG, wherein the antibodies are IgG antibodies, IgA antibodies, or a combination thereof. The invention further permits discriminating between IgG and IgA antibodies which specifically recognize tTG. Likewise, in one embodiment, the invention facilitates detection of the presence or absence of antibodies that specifically recognize deamidated gliadin, wherein the antibodies are IgG antibodies, IgA antibodies, or a combination thereof The invention further permits discriminating between IgG and IgA antibodies which specifically recognize deamidated gliadin

The method of the invention is suitable for performing on a biological sample obtained from an individual of any age or gender. In one embodiment, the individual has been determined to be or is suspected of being IgA deficient. The method may be performed once, or a series of tests may be performed to, for example, monitor an individual's response to a treatment, such as a modification in diet.

In one embodiment, the invention comprises fixing the result of performing the method of the invention in a tangible medium of expression, such as a digitized computer record. The invention further comprises communication the result of the performing the method of the invention to a health care provider.

The invention also provides kits comprising a chimeric protein of the invention and a solid matrix to which the chimeric protein is or can be affixed. The kits may further comprise components for biological sample collection and reagents for antibody detection, positive controls, and the like. The kits may also include instructions for using the kits.

Also provided are DNA polynucleotides encoding the chimeric proteins of the invention. In one embodiment, the DNA polynucleotide comprises a sequence encoding the amino acid sequence of the chimeric protein depicted in FIG. 1. The DNA sequence (and therefore the chimeric proteins of the invention) may or may not include a sequence encoding the MGSHHHHHH (SEQ ID NO:3) amino acid sequence at the N-terminus of the protein depicted in FIG. 1.

The invention is further illustrated by the following Examples and Figures, which are intended to exemplify but not limit the invention.

### EXAMPLE 1

This Example provides in FIG. 1 a graphical illustration of a DNA construct encoding a chimeric protein provided by the invention, as well as an amino acid sequence of a representative chimeric protein that is suitable for use in the method of the invention. The amino acid sequence is presented as SEQ ID NO:1.

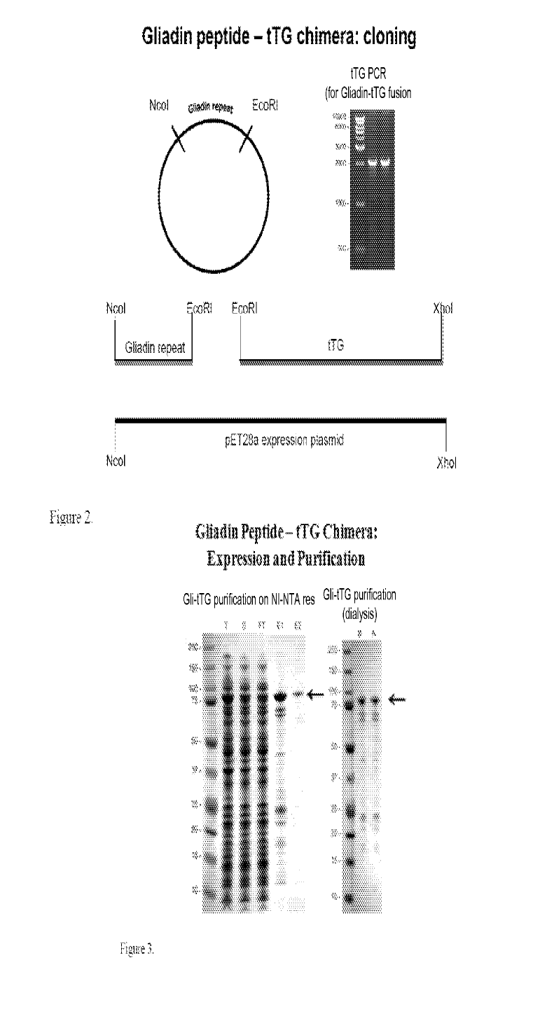

FIG. 2 provides a graphical depiction of a cloning scheme for a chimeric protein of the invention, as well as a photographic representation of an electrophoretic analysis of a recombinant expression vector suitable for making a chimeric protein of the invention. FIG. 3 provides a photographic representation of the expression and purification of a chimeric protein of the invention. The representative chimeric protein analyzed in FIG. 3 consists of the amino acid sequence shown in FIG. 1.

### EXAMPLE 2

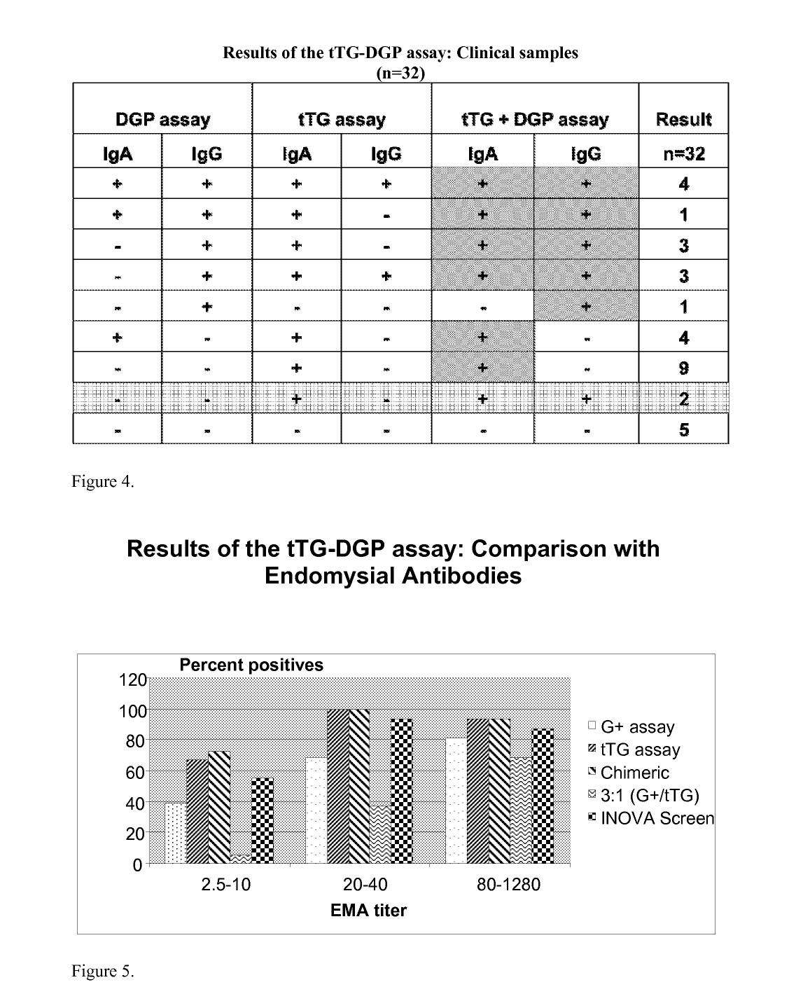

This Example illustrates analysis of clinical samples from subjects with CD to detect IgA and IgG antibodies that recognize a chimeric protein having the amino acid sequence depicted in FIG. 1. Results from analysis of 32 different clinical samples are presented in FIG. 4. As can be seen from FIG. 4, in some confirmed celiac patient samples, IgG detection is below threshold for determining a positive result using deamidated gliadin and tTG separately, but is positive for IgG when the chimeric protein is used for determining antibodies. (e.g., compare the results presented in second row from bottom of FIG. 4.) This demonstrates that the invention facilitates improved CD diagnosis, relative to using deamidated gliadin and tTG that are not provided as a fusion protein. This enhanced sensitivity of the current invention may be attributable to the generation of neo-epitopes that are unique to the complex in the chimeric tTG-DGP fusion. These epitopes may not be present or accessible in the individual tTG and/or DGP molecules.

### EXAMPLE 3

This Example illustrates an embodiment of the invention wherein the chimeric protein comprises 5 deamidated glaidin repeats.

To obtain the data presented in this Example, we performed additional experiments using chimeric protein comprising one molecule of tTG fused with five repeats of deamidated gliadin peptide. We demonstrate that, relative to chimeric proteins comprising one or three deamidated gliadin peptide repeats, chimeric proteins comprising tTG and five deamidated gliadin peptide (DGP) repeats have improved positive and negative predictive values when used compared to a combination of individual assays for detecting IgA and IgG antibodies against tTG and DGP respectively.

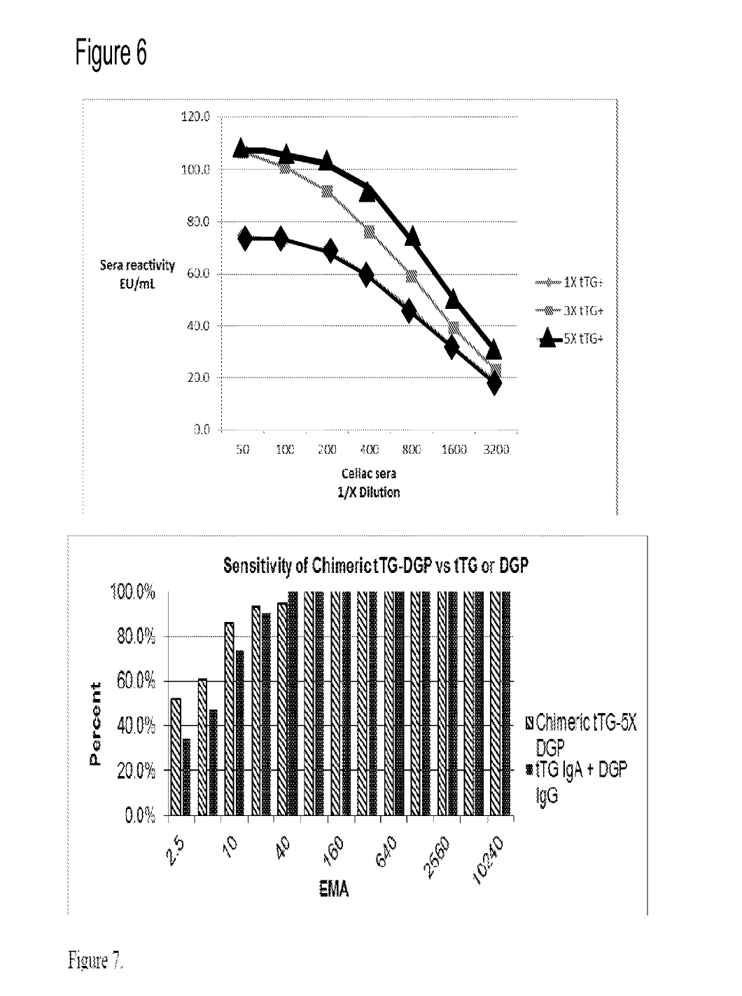

FIG. 6 summarizes EU values (relative ELISA units) of positive sera when reacted with an ELISA plate containing tTG+1 or 3 or 5 copies of the DGP. It will be recognized by those skilled in the art that analytical sensitivity of tTG+5X DGP is clearly superior to tTG+3xDGP and tTG+1x DGP over a wide range of serum dilutions.

Several CD sera positive for EMA (i.e., positive for endomycial antigen as determined by immunohistochemical staining for EMA reactivity on the smooth muscle tissue on the primate distal oesophagus sections) staining with known titers (a serial dilution point at which a positive sera positive on tissue sections gives negative reaction) are assayed on both the chimeric tTG-5XDGP screen ELISA for IgA and IgG and individual tTG (IgA) and DGP (IgG) assays.

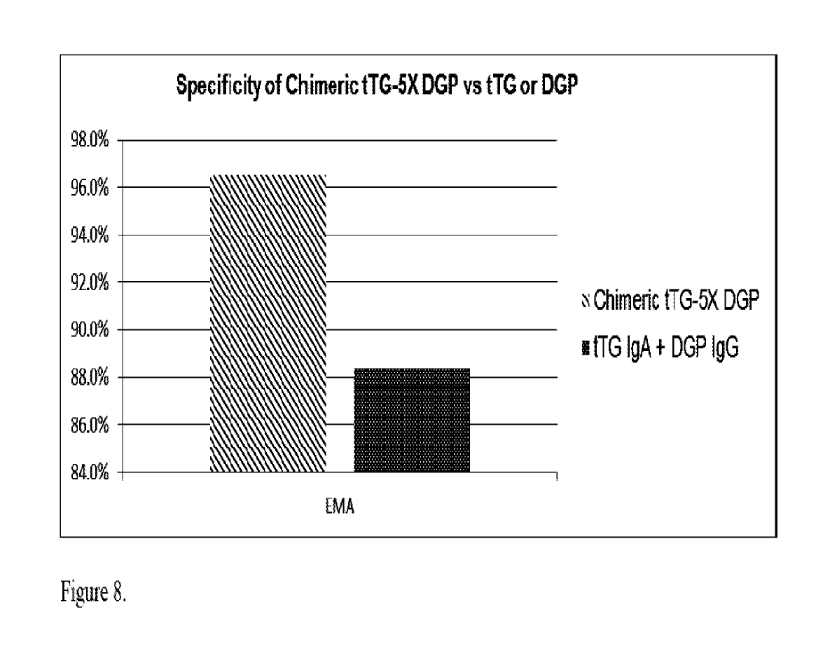

The percent sera that are accurately identified were plotted against EMA titer and the results are presented in FIG. 7. The data show that the sensitivity of chimeric antigen is clearly superior for EMA titer range of 2.5 to 40 where the sensitivity of selected assay has the most influence on the CD detection and patient diagnosis. An assay with higher sensitivity is required to detect these sera with low levels of autoantibodies. The specificity of chimeric tTG-5X dGP was compared to individual assays by testing 144 EMA negative samples on both assays (FIG. 8). ELISA using the chimeric (5X) antigen demonstrated a better specificity compared to the combined specificity of the individual ELISAs for IgG and IgA. Thus, the tTG-5X-DGP chimeric protein of the invention provides a better alternative for the detection of celiac positive sera with an overall improved positive and negative predictive value compared to the combination of two individual celiac immunoassays, reducing the amount of required time, labor and costs.

The foregoing examples are intended to illustrate the invention. Those skilled in the art will recognized that minor modifications can be made without deviating from the spirit of the invention.

We claim:

1. A method for diagnosing celiac disease in an individual comprising contacting a biological sample obtained from the individual with a protein, wherein the protein comprises the amino acid sequence of tissue transglutaminases (tTG) and the amino acid sequence of deamidated gliadin, wherein the amino acid sequence of the deamidated gliadin is repeated in the chimeric protein at least three times, determining the presence or absence of antibodies in the biological sample that specifically recognize the chimeric protein, and identifying the individual as having celiac disease based on the presence of the antibodies in the biological sample that specifically recognize the chimeric protein, or identifying the individual as not having celiac disease based on the absence of the antibodies that specifically recognize the chimeric protein.

2. The method of claim 1, wherein the absence of IgG and IgA antibodies that recognize the chimeric protein is indicative that the individual does not have celiac disease.

3. The method of claim 1, wherein the amino acid sequence of deamidated gliadin is repeated in the chimeric protein five times.

4. The method of claim 1, wherein the individual is IgA deficient and is identified as having celiac disease.

5. The method of claim 3, wherein the individual is IgA deficient and is identified as having celiac disease.

6. The method of claim 1, wherein the determining the presence or absence of antibodies in the biological sample is performed by enzyme-linked immunosorbent assay (ELISA).

7. A composition comprising a chimeric protein, wherein the chimeric protein comprises the amino acid sequences of tissue transglutaminases (tTG) and the amino acid sequences of deamidated gliadin, wherein the amino acid sequence of the deamidated gliadin is repeated in the chimeric protein.

8. The composition of claim 7, wherein the amino acid sequence of the deamidated gliadin is repeated in the chimeric protein at least three times.

9. The composition of claim 8, wherein the amino acid sequence of the deamidated gliadin is repeated in the chimeric protein five times.

10. A composition comprising antibodies that have been isolated from an individual, wherein the antibodies are complexed to a protein, wherein the chimeric protein comprises the amino acid sequences of tissue transglutaminases (tTG) and the amino acid sequences of deamidated gliadin, wherein the amino acid sequence of the deamidated gliadin is repeated in the chimeric protein.

11. The composition of claim 10, wherein the antibodies that have been isolated from the individual are IgA antibodies.

12. The composition of claim 10, wherein the deamidated gliadin is repeated in the chimeric protein at least three times.

13. The composition of claim 10, wherein the deamidated gliadin is repeated in the chimeric protein at least five times.

14. The composition of claim 10, wherein the individual is identified as having celiac disease.


SEQUENCE LISTING (3 sequences)

SEQ ID NO: 1 - protein, 739 aa, artificial sequence
  Chimeric protein comprising tTG and deamidated gliadin peptide sequence 3x
MGSHHHHHHSSGPLQPEQPFPGSGPLQPEQPFPGSGPLQPEQPFPGSGDTSEFAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 2 - protein, 9 aa, human
PLQPEQPFP

SEQ ID NO: 3 - protein, 9 aa, artificial sequence
  HIS tag sequence
MGSHHHHHH